(19) United States
(12) Plant Patent Application Publication
(10) Pub. No.: US 2018/0303017 P1
(43) Pub. Date: Oct. 18, 2018

(54) Blackberry Plant Named 'Valentina'

(50) ;

(71) Applicant: Hortifrut S.A., Santiago (CL)

(72) Inventors: Maria Pilar Banados, San Fernando (CL); Natalia Torres, Talca (CL)

(73) Assignee: Hortifrut S.A., Santiago (CL)

(21) Appl. No.: 15/731,096

(22) Filed: Apr. 17, 2017

(51) Int. Cl. A01H 6/74

(52) U.S. Cl. PLT/203

(57) ABSTRACT

‘Valentina’ is completely thornless, it has a high degree of primocane fruiting expression and high productivity on both primocanes and floricanes. Additionally, ‘Valentina’ also produces firm and sweet berries that are of a desirable medium size (8 to 11 g) with a very pleasant flavor. The high sugar content of the berries of ‘Valentina’ is well balanced with titratable acidity (13.7° brix with 0.9% acidity) and the receptacle is very soft, giving a melting sensation in the mouth.

Botanical name of the claimed plant: Family: Rosaceae. Botanical name: Rubus subgenus Rubus Watson Common name: Blackberry.

Cultivar name of the claimed plant: ‘Valentina’.

### BACKGROUND AND SUMMARY

Valentina is a novel, medium to large size (8 to 10 g) blackberry that produces fruit with high sweetness on both floricanes and primocanes in Hijuelas (fifth region of Valparaiso), Central Chile. Plants of ‘Valentina’ are thornless and of medium vigor. The plants are easy to grow and manipulate owing to their thornless nature and good growth habit.

The main attributes of ‘Valentina’ fruit are: high firmness, a very soft receptacle that can be described as “melting” and which imparts an excellent mouthfeel while eating the fruit. Fruit of ‘Valentina’ is sweet and has no discernable bitter aftertaste. On a scale of 1 to 5 (with 5 being the firmest), fruit of ‘Valentina’ ranked an average of 4.5 for tactile assessment of firmness. However, fruit of ‘Valentina’ does show some post-harvest regression to red druplets, with 1 to 4 druplets turning red after 5 days in cold storage. This is a low rate of regression as compared to other blackberry varieties.

There are very few differences between fruits from floricanes and primocanes with respect to flavor, firmness, and mouthfeel. However, differences in fruit quality attributes are observed between plants grown under plastic tunnels as compared to growing in open air. The floricane crop of ‘Valentina’ begins to ripen in the first week of December in Hijuelas (fifth region of Valparaiso), Central Chile, when grown under plastic tunnels. Plants grown in open field conditions begin ripening in the third week of December in this same region. For plants grown in plastic tunnels in this same region, the primocane crop of ‘Valentina’ begins ripening between the third and fourth week of January in this region, and continues production until week 12. In order to maximize yields on primocanes of ‘Valentina’, it is necessary to prune out the apical 10 to 20 cm of the shoots of the actively-growing primocanes once these reach 80 to 100 cm in height. After such pruning, lateral buds begin to grow 7 to 10 days later. The laterals begin to flower 50 to 60 days after the tip pruning. The chill requirement for spring budbreak of floricanes of ‘Valentina’ is estimated to be 300 hours below 7° C. This would categorize ‘Valentina’ as a “low-chill” variety, as compared to other blackberry cultivars. Primocanes can flower and fruit without any chill hours at all.

All data and observations presented herein were made during the 2014/15 southern hemisphere summer on plants growing at Hijuelas, 5^{th} Region of Valparaiso which province is located in the Central part of Chile. The coordinates of the trial site are: 32° 54′11″S; 71° 5′40″W, and the site is located at 330 meters above sea level. Chilling hours for this site are counted between May and June of each year, and range between 400 to 500 chilling hours in open field conditions.

‘Amara’ (U.S. Plant Pat. No. 26,413), ‘APF-153T’ (U.S. Plant Pat. No. 26,990), ‘APF-77’ (U.S. Plant Pat. No. 24,249), and ‘Camila’ (U.S. Plant Pat. No. 26,328) are similar cultivars in that they all show primocane fruiting habit. ‘Amara’, ‘APF-153T’, and ‘Valentina’ are further similar in that they are also thornless and with high fruit firmness.

As compared to the blackberry cultivar ‘Amara’, ‘Valentina’ is much more productive on its primocane crop. One year old plants of ‘Valentina’ had an average yield of 3.4 kg of fruit, per meter of row on primocanes versus an average of 0.7 kg per meter of row on primocanes of ‘Amara’. Furthermore, the fruit of ‘Valentina’ ripens earlier than that of ‘Amara’ and is sweeter, with higher levels of aromatics (which impart a fruity flavor) and with lower acidity than fruit of ‘Amara’. Another difference is that the receptacle of the fruit of ‘Valentina’ is softer than that of ‘Amara’ which results in a more pleasurable mouthfeel of the former cultivar.

As compared to ‘Camila’, ‘Valentina’ is different in that it has thornless stems, whereas ‘Camila’ canes are thorny. Fruits of ‘Valentina’ are also firmer than those of ‘Camila’. On a relative scale of 1 to 5, with 5 being the firmest, the average fruit firmness of ‘Valentina’ was 4.5 versus 3.4 for the fruit of ‘Camila’. The difference in fruit firmness between these two varieties becomes more notable post-harvest: after one week of storage at 4° C., 70% of the fruit of ‘Valentina’ were classified in the “firm” or “very firm” category, whereas in ‘Camila’ only 10% of the fruit were classified as being “firm” or “very firm”. ‘Valentina’ also has less post-harvest reversion to red druplets. On the other hand, the primocane crop of ‘Valentina’ begins ripening later than that of ‘Camila’.

As compared to ‘APF-153T’, ‘Valentina’ produces fruit that is slightly smaller in weight and has a higher chilling requirement. Primocane base color of APF-153T is red-purple versus yellow-green for ‘Valentina’. Postharvest storage potential of ‘Valentina’ exceeds that of APF-155T in that the former has firmer fruit and lower rates of post-harvest drupelet color reversion.

‘Valentina’ has many desirable attributes: It is completely thornless, it has a high degree of primocane fruiting expression and high productivity on both primocanes and floricanes. Additionally, ‘Valentina’ also produces firm and sweet berries that are of a desirable medium size (8 to 11 g) with a very pleasant flavor. The high sugar content of the berries of ‘Valentina’ is well balanced with titratable acidity (13.7° brix with 0.9% acidity) and the receptacle is very soft, giving a melting sensation in the mouth.

### BRIEF DESCRIPTION OF THE PHOTOGRAPHS

FIG. 1 is a photograph that shows primocane fruits of ‘Valentina’ during their ripening stage. Image taken on 3 Feb. 2015 (week 4) in Hualcapo, fifth Region of Valparaiso, Chile.

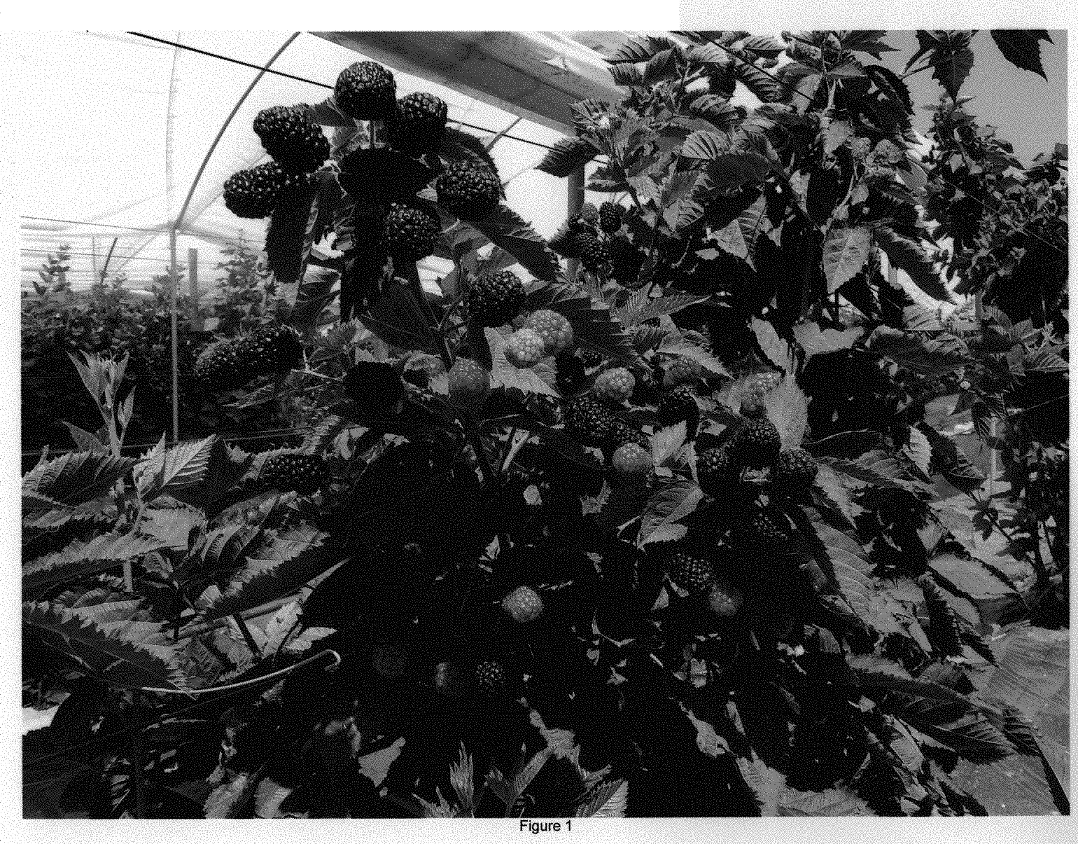

FIG. 2 is a photograph that shows the original plant of ‘Valentina’ which was planted in Hualcapo, Fifth Region of Valparasio, Chile in July 2013. This plant was therefore 1.5 years old when the photograph was taken on 3 Feb. 2015.

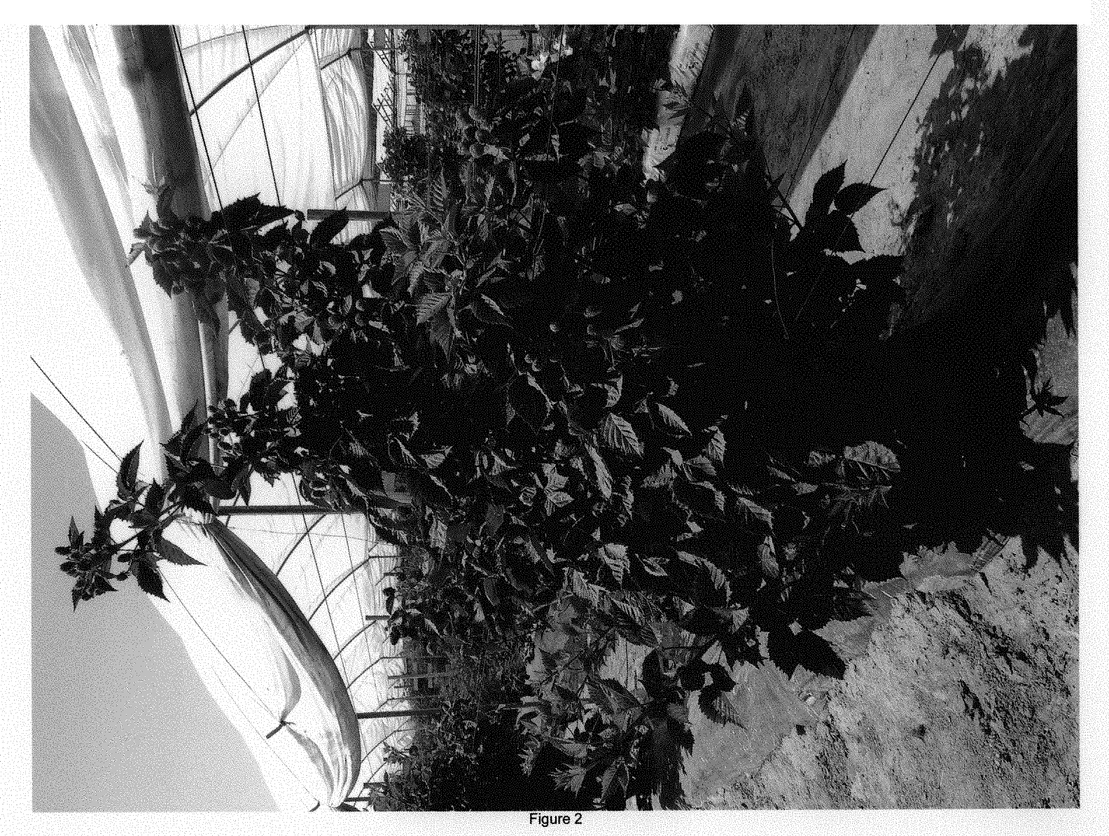

FIG. 3 is a photograph that shows primocane fruits of ‘Valentina’ which were harvested on 5 Feb. 2015 from plants growing in Hualcapo, Fifth Region of Valpariaiso, Chile. The photograph was taken the same day of harvest.

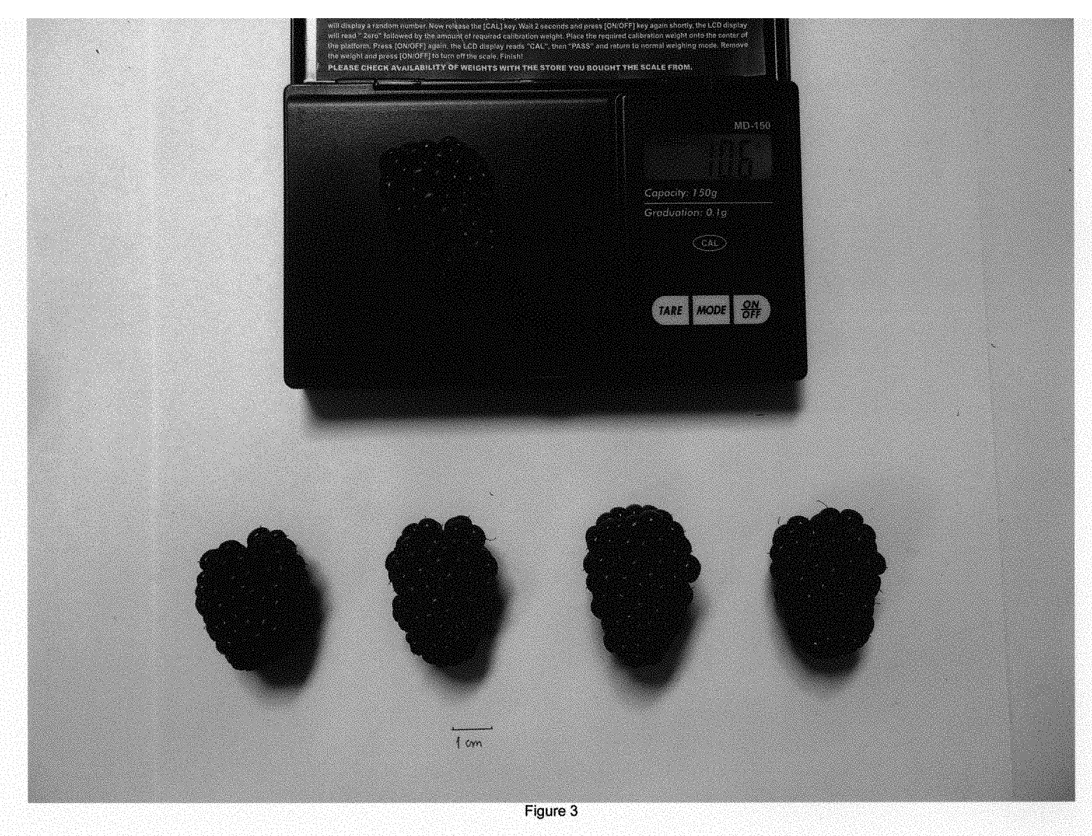

### DETAILED DESCRIPTION

Note: statements of characteristics herein represent exemplary observations of the cultivar herein and will vary depending on time of year, location, annual weather, etc. Where dimensions, sizes, colors, and other characteristics are given, it is to be understood that such characteristics are approximations and averages. For the purposes of this application, data for ‘Valentina’ was collected from both the original plant and from a trial plot consisting of 10 plants growing in Hualcapo, Fifth region of Valparasio, Chile during 2 consecutive growing seasons (2014-15 and 2015-16).
Cultivar name: ‘Valentina’.
Classification:
   Family.—Rosaceae.
   Botanical name.—Rubus subgenus Rubus Watson.
   Common name.—Blackberry.
Parentage:
   Female parent.—A-2445, an unpatented, proprietary selection from University of Arkansas that was never released commercially and was discarded (e.g. no longer exists) in 2013. The female parent differed from the instant cultivar in that it was neither primocane-fruiting, nor was it thornless.
   Male parent.—APF-186T, an unpatented, proprietary selection that was never released commercially and no longer exists, as it was discarded in 2014. It was similar to ‘Valentina’ in that it had primocane-fruiting habit as well as thornlessness. However, it differed in that it had smaller fruit and was less productive.
Further information about the new variety amara: ‘Valentina’ was the result of a controlled hand pollination made near Clarksville, Ark., USA in 2010 between the female parent ‘A-2445’ and the male parent being ‘APF-186T’. Seeds from this controlled pollination were then sent to a nursery in Hijuelas, Fifth Region of Valparasio, Chile in September 2010. Seeds of this cross were germinated in a greenhouse between November 2010 and March 2011 and planted into the seedling field in the vicinity of Hualcapo, Fifth Region of Valaparaiso, Chile, which is located at 32° 54′11″S, 71° 5′40″W at an elevation of 330 meters above sea level. The seedlings fruited for the first time in the southern hemisphere summer of 2013 on primocanes and the seedling that would become ‘Valentina’ was selected on Jan. 25, 2013 for its outstanding fruit firmness and good flavor. References to color refer to the R.H.S.—Fifth Edition (2007). ‘Valentina’ was first asexually propagated in March 2014 in micropropagation (in vitro). Then in June 2014 it was propagated by division of the original seedling mother plant and by etiolated shoots from root cuttings. All methods of asexual reproduction used to date for the instant variety have yielded plants that were true-to-type in successive generations of asexual reproduction. The plants used in the descriptions were planted between 2014 and 2015, and were growing in Hualcapo, Fifth Region of Valparaiso, Chile. All plants used in the descriptions were grown in open field conditions (i.e. not under plastic culture) with the growing primocanes being tip-pruned between October and December of each year. Tips were pruned with the primocanes reached about 80 cm in height, the distal 20 to 30 cm of the primocanes was removed during the pruning.
General description:
A) Plant: Plants of ‘Valentina’ are thornless with strong primocane-fruiting habit, moderate vigor and above-average productivity. In Central Chile, the floricane harvest of ‘Valentina’ begins in week 51 (mid-December) and the primocane harvest begins in week 4 on primocanes. The fruit quality is very good, with high sugar content, good flavor and a high level of firmness. Field observations were made between 2013 and 2015 in Hualcapo, Fifth Region, Chile, including evaluation over three primocane fruiting cycles and 2 floricane fruiting cycles. ‘Valentina’ consistently had high productivity on both floricanes and primocanes, along with high fruit firmness.
   Growth.—‘Valentina’ is moderately vigorous and has a semi-erect growth habit. Primocanes are abundant and emerge both from the crown of the plant as well as roots as suckers.
   Growth rate.—The average height of un-tipped canes is 1.8 meters, and they arrive at this height within four months of emergence from the soil in the spring.
   Productivity.—‘Valentina’ plants are highly productive. The productivity was measured during the 2015-16growing season on plants growing in a trial plot located in the vicinity of Hualcapo, Fifth Region of Valparaiso, Chile. The floricane crop averaged 2.7 kg fruit per linear meter of row, and the primocane crop averaged 3.4 kg of fruit per linear meter of row. This is considered high productivity.
   Cold hardiness.—Ultimate cold hardiness is unknown, but in Chile dormant plants have resisted midwinter lows of −3° C. without damage.
   Branching height of the plants.—Plants of ‘Valentina’ do not normally branch on primocanes unless they are tip-pruned. When the primocanes are tip-pruned to 60cm in height during the spring, laterals emerge along the entire length of the cane and these can reach a length of 130 cm.
B) Canes:
General description: Canes are thornless, semi-erect and with medium vigor. The plants produce abundant quantities of primocanes.
   Cane diameter (indicate point of measurement).—Floricane: Base: 1.78±0.17 cm Midpoint: 1.45±0.02 cm Terminal: 0.089±0.10 cm Immature primocane: Base: 0.96±0.16 cm Midpoint: 0.64±0.10 cm Terminal: 0.4±0.07 cm Mature primocane: Base: 1.81±0.26 cm Midpoint: 1.27±0.08 cm Terminal: 0.56±0.09cm.
   Cane length.—The figures here were for plants growing in soil, but under a plastic tunnel (macrotunnel) Floricanes: 1.9 meters if not pinch-pruned Mature primocanes: 1.9 meters, if not pitch-pruned, though primocanes are normally pruned to 60 cm in height.
   Thorns.—Totally absent.
   Cane texture.—Floricanes: smooth surface without thorns Immature primocane: pilose Mature primocane: pilose Internode length: Base: 5.56±0.9 cm Midpoint: 4.90±1.1 cm Terminal: 3.90±1.7 cm Thorn density/30 cm; this cultivar has no thorns, therefore, the density is: Base: 0 Midpoint: 0 Terminal: 0 Primocane color: Base: Background Color: Yellow-Green Group 146 C, with overtones in the Greyed-red Group 181 A Midpoint: Background color is Yellow-green Group 146 C, with overtones in the Greyed-red Group 178 A-B. Terminal: Background color is Yellow-green Group 144 A-B with overtones in the Greyed-red Group 178 A-B Floricane color: Base: Background color is Yellow-green Group 147 B, with overtones in the Greyed-orange Group 167 D. Midpoint: Background color is Yellow-green Group 147 C, with overtones in the Greyed-orange Group 164 B Terminal: Background color is Green group 143 C with overtones in the Orange-red Group 34 A-B Date of primocane emergence: In Hualcapo, Fifth Region of Valparaiso, Chile, primocanes emerge on about week 38 (southern hemisphere spring) and continue emerging in flushes for 4 to 10 weeks until week 48, depending on pruning management and nitrogen fertilization. For example, if left unpruned, new primocanes are observed until week 48. Date of budbreak: Vegetative budbreak on floricanes in the spring occurs beginning in week 33 in Hualcapo, Fifth Region of Valparaiso, Chile. Number of new canes: A well-established plant (i.e. one that has been in the ground for 2 or 3 years) with emit an average of 7-9 new primocanes/plant each year. Number of branches on dormant canes: If left unpruned, a primocane (which later becomes a floricane once it goes dormant) will not branch naturally. However, in practice, the primocanes are always pinch-pruned at approximately 60 cm in height to force lateral branching, which results in an average of 6 branches. Buds on dormant cane: Shape: acuminate Length: 0.8 cm Width: Base: 0.30 cm Midpoint: 0.24 cm Terminal: 0.11 cm Color: Greyed-red Group 178 A-B.
   Predominate distribution of branches.—The branches (while stimulated by pinch-pruning) are distributed along the entire length of the cane.
C) Foliage:
General description:
   Complete leaf.—Width: 20.7±1.45 cm Length: 19.8±2.80 cm (including petiole) Number of leaflets: 5 Shape: Palmate.
   Leaflet (without petiolules).—Width: 6.9±0.86 cm Length: 9.8±0.97 cm (including petiolules) Margin: thrice serrate Base: cordate Apex: broadly acuminate Shape: Ovate. Surface: Pubescent of whitish color concentrated mainly around the veins on the abaxial side of the leaf Color: Base Adaxial: Green Group N137 B Base Abaxial: Green Group 147 B Midpoint Adaxial: Green Group N137 A-B Midpoint Abaxial: Green Group 147 B Terminal Adaxial: Green Group 146 A Terminal Abaxial: Green Group 147 B Veins Adaxial: Green Group 146 B-C Veins Abaxial: Green Group 146 C-D Pubescence density: moderate Pubescence color: (Green Group 143 D).
   Petioles.—Length: 7.1±0.67 cm Color: Greyed-Red Group 178A Diameter: 0.21±0.08 cm Texture: puberulent.
   Petiolules.—Length: 1.23±1.2 cm Color: Greyed-Red Group 178a Diameter: 0.10±0.04 cm Texture: puberulent.
   Stipules.—Length: 0.1±0.03 cm Width: 0.01 cm Texture: Puberulent Color: Yellow-Green Group 146 B.
Flowers:
   General description.—Flowers of ‘Valentina’ originate both on fruiting laterals of floricanes (like the majority of blackberry genotypes) but also in current-season shoots (e.g. primocanes). Floral morphology is very similar in the fruiting laterals of both floricanes and primocanes, however, early primocanes tend to have larger flowers than later primocanes. Flowers of ‘Valentina’ are perfect, self-fertile and have 5 white petals, numerous stamens and pistils, pollen is abundant and is a self-fertile flower. Flowers are very attractive to bees, and no misshapen flowers have been observed at any point during the season.
D) Flowers foliage:
   Primocane.—Date of bloom: (Central Chile) 10% bloom: week 49 50% bloom: week 51 Last bloom: week 1. Floricane: Date of bloom: (Southern Hemisphere) 10% bloom: week 42 50% bloom: week 42 Last bloom: week 46. Petal color: White Group NN155 B-C Reproductive organs: Stamens: erect, 102 to 128 stamens per flower, 0.18 to 0.31 mm Pistils: abundant, 120 to 130 per flower Pollen: pollen is plentiful and self-fertile Ovary: Superior. Flower diameter: 3.1±2.5 cm Petal size: Width: 1.15±0.21 cm Length: 0.89±0.0.09 cm Unopened buds: Shape: Rounded Length: 1.0±0.2 cm Width: 08±0.09 cm Color: Green Group 138A Pedicles: Length: 4.5±1.5 cm Width: 0.1±0.05 cm Color: Green Group 138A Texture: Pubescent Fragrance: none Average number flowers per lateral/cluster: 10 to 15 Average number of petals per flower: Peduncle length: 7.0±2.1 cm Peduncle diameter: 0.4±0.05 cm Peduncle color: Green Group 138A Peduncle texture: Pubescent.
E) Fruit foliage:
General description: ‘Valentina’ is a medium sized (8 to 11 g), sweet blackberry that produces fruit in both floricanes and primocanes in Hualcapo, Fifth Region of Valparaiso, Chile. The main attributes of the fruit are their firmness, very pleasant eating experience with a very soft receptacle that presents as a melting sensation in the mouth with none of the bitter aftertaste that is common in other blackberry genotypes. Firmness of the fruit ranks an average of 4.5 on a 1 to 5 scale, with 5 being the firmest fruit. ‘Valentina’ fruit shows some amount of post-harvest color regression with 1 to 4 drupelets per fruit turning red after 5 days in cold storage, which we qualified as a “low” rate of color regression compared to most blackberry genotypes. Floricane and primocane fruits of ‘Valentina’ are essentially indistinguishable, but differences in fruit quality attributes can occur from different growing conditions, (i.e. when plants are grown either under plastic tunnel or in open air). The floricane crop ripens from the first week of December when grown under plastic tunnels, or two weeks later, if grown in open air. Fruits of pruned ‘Valentina’ primocanes begin ripening from week 3 or 4 of the year under tunnels, continuing until week 12. Primocanes of ‘Valentina’ need one pinch- or tip-pruning when primocanes reach 80 to 100 cm in height, the pruning should the distal 10 to 20 cm of shoot. Laterals then start to grow 7 to 10 days after pruning and produce flowers within 50 to 60 days, post pruning. ‘Valentina’ requires about 300 chilling hours to initiate budbreak on floricanes, this means it could be regarded as having a “low” chilling requirement relative to other blackberry genotypes. Of course, there is no chilling needed for the primocane budbreak (and cropping). Chilling hours for the evaluation site were recorded only for the time period between May and June and these range between 400 to 500 hours in open field (i.e. not under tunnels) conditions.
   Primocane.—Average first ripe date: Primocane crop starts on about week 4 at the Hualcapo trial site and lasts for 40 to 50 days (depending on primocane tipping height). ‘Valentine’ primocanes begin cropping 2 weeks after those of ‘Camila” but 3 weeks before those ‘Amara’. Size: medium/large (11.9 g, on average) Diameter: Equator: 23.84 mm Base pole: 16.5 mm Terminal pole: 22.2 mm Length: 32.5 mm Shape: oblong Drupelet size: Medium (5.49 mm) Drupelet number 100-120 Drupelet skin color: Black Group 203 Drupelet flesh color: Greyed-Purple Group N186 Seed Color: Black Group 203 Seed size: medium to large Seed diameter: 2.1 mm Seed length: 4.3 mm Firmness: very high, rated 4.5 on a scale from 1 (soft) to 5 (very firm) Flavor: Fruit is similar to floricane, sweet very flavorful, aromatic, and no bitterness Soluble solids: 10.5 to 14.2° brix depending on ripening stage and frequency of harvest pH: 3.3 to 3.7 Acidity: 0.7 to 0.9% Yield potential: 10.4 tons/ha Processed quality: no drupelet loss or color reversion was observed in individually quick frozen (IQF) fruit, thus high quality processed fruit for IQF. Uses: Fresh Market.
   Floricane.—Average first ripe date: week 51, the fruiting period lasting about 60 days Size: Medium (10.8 g on average). Diameter: Equator: 26.1 mm Base pole: 21.9 mm Terminal pole: 23.65 mm Length: 3.40 cm Shape: oblong Drupelet size: Medium, (5.3 mm) Drupelet number: 100-120 per fruit Drupelet skin color: Black Group 203 Drupelet flesh color: Greyed-Purple Group N186 Receptacle color: white Seed color: Black Group 203 (for clean, dry seed) Seed size: medium to large, seed shape is flat and elongated Diameter: 1.98 mm Length: 4.1 mm Firmness: fruit is firm, rated 4.5 (in a 1 to 5 scale) Flavor: fruit is sweet with a tart finish, but without bitter aftertaste and having an intense fruity flavor. Additionally, the receptacle is soft, such that it “melts” in the mouth. Soluble solids: 10.7 to 14.2° brix depending on harvest frequency and ripening pH: 3.3 to 3.7. Acidity: 0.7 to 0.9% Yield potential: 13.6 tons/ha Processed quality: no drupelet loss or color reversion was observed in individually quick frozen (IQF) fruit, thus high quality processed fruit for IQF Uses: Fresh Market
Resistances and tolerances: ‘Valentina’ shows some susceptibility to redberry mite. It also has low resistance to sunburn on the fruit, presenting white to light-brown drupelets when exposed to full summer sunlight. Production under tunnel or shading net is therefore recommended for this genotype.

What is claimed is:

1. A new and distinct cultivar of Blackberry plant named ‘Valentina’ as described and shown herein.
